(19) United States
(12) Patent Application Publication
(10) Pub. No.: US 2023/0400430 A1
(43) Pub. Date: Dec. 14, 2023

(54) ELECTROCHEMICAL SENSOR FOR SIMULTANEOUS DETECTION OF DOPAMINE AND SEROTONIN AND MANUFACTURING METHOD THEREOF

(71) Applicant: KOREA INSTITUTE OF SCIENCE AND TECHNOLOGY, Seoul (KR)

(72) Inventors: Yi Jae LEE, Seoul (KR); Seung Hyeon KO, Seoul (KR)

(73) Assignee: KOREA INSTITUTE OF SCIENCE AND TECHNOLOGY, Seoul (KR)

(21) Appl. No.: 18/302,588

(22) Filed: Apr. 18, 2023

(30) Foreign Application Priority Data
Jun. 14, 2022 (KR) 10-2022-0072320

(51) Int. Cl. G01N 27/327; G01N 27/48; C25D 13/02; C25D 13/12; A61B 5/1468

(52) U.S. Cl. CPC G01N 27/3275; G01N 27/48; C25D 13/02; C25D 13/12; A61B 5/1468; A61B 2562/125

(57) ABSTRACT

The present disclosure relates to an electrochemical sensor for simultaneous detection of dopamine and serotonin including an electrode containing a reduced graphene oxide (rGO), poly(3,4-ethylenedioxythiophene):poly(4-styrenesulfonate) (PEDOT:PSS), and Nafion, wherein the sensor has high interfacial conductivity and electrocatalytic properties and further improves the negatively charged electrode interface, thereby enabling high sensitivity selective measurement of dopamine and serotonin. In addition, since the sensor according to the present disclosure is stable for a long time and has high reproducibility, it can be used for clinical diagnosis of various brain and neurological diseases, drug treatment, biological research using changes in the concentration of neurotransmitters, and biochip application fields.

### CROSS-REFERENCE TO RELATED APPLICATION(S)

This application claims the benefit of Korean Patent Application No. 10-2022-0072320, filed on Jun. 14, 2022, in the Korean Intellectual Property Office, the entire disclosure of which is incorporated herein by reference for all purposes.

### BACKGROUND

### 1. Field of the Invention

One or more example embodiments relate to an electrochemical sensor for simultaneous detection of dopamine and serotonin, a manufacturing method thereof, etc.

### 2. Description of the Related Art

Since neurotransmitters not only maintain a nerve function, but also has an important effect on various nervous systems that interact between brain and nerves, dysregulation or imbalance of the neurotransmitters may cause several physiological and psychological diseases. Since dopamine (DA) and serotonin (5-hydroxytryptamine, 5-HT) among them are monoamine neurotransmitters widely distributed in the human body and brain, and play a role in regulating numerous behavioral and physiological functions, deficiency of these may cause mood-related disorders, depression, migraines, sexual disorders, Parkinson's disease, or the like. Therefore, monitoring of dopamine and serotonin is very important for the diagnosis of various diseases.

Nevertheless, most of the neural probes mainly used in the field of neuroscience or clinical are only focused on electrical nerve stimulation and neural signal recording, and there is a lack of tools for monitoring multiple neurotransmitters that are highly correlated with brain function regulation and brain disease causing.

Conventional methods for detecting neurotransmitters include high performance liquid chromatography (HPLC), liquid chromatography-electrospray tandem mass spectrometry (LC-ES tandem MS), surface enhanced Raman scattering spectroscopy (SERS), fluorescence, and the like. Although these methods are quite efficient, there are limitations in that detection time is long, a large amount of sample is required, sampling is complex, and expensive and bulky instruments are included. Therefore, it is essentially required to develop a miniaturized, high-sensitivity sensing electrode that can be utilized in monitoring of multiple neurotransmitters by being applied to a neural probe.

In order to overcome these limitations, an electrochemical detection method capable of simultaneous quantitative analysis with high sensitivity has been proposed. However, serotonin is difficult to distinguish from dopamine having a similar structure, and it is not easy to detect it since it is disturbed by interfering substances such as ascorbic acid (AA) and uric acid (UA). In addition, the detection of serotonin in physiological samples is negatively affected by electroactive metabolites so that there is a problem in that both selectivity and sensitivity are low.

Meanwhile, graphene oxide (GO) has attracted attention in various application fields since it can increase the sensitivity of the sensor as a catalyst support due to its high specific surface area and unique structure. GO has a unique ability to act as a versatile dispersant, has high biocompatibility, and has excellent electrocatalytic properties. However, the oxygen-functional groups in the GO sheet can be dispersed and readily soluble in water and other solvents so that there is a high possibility of causing instability in the analysis of real samples.

Accordingly, the present inventors obtained a reduced graphene oxide (rGO) through economical and environmentally friendly electrochemical reduction, and completed present disclosure by confirming that an electrochemical sensor containing the reduced graphene oxide (rGO), poly(3,4-ethylenedioxythiophene):poly(4-styrenesulfonate) (PEDOT:PSS), and Nafion is capable of simultaneously detecting dopamine and serotonin selectively and highly sensitively.

### SUMMARY

Example embodiments provide a working electrode containing a reduced graphene oxide (rGO), PEDOT:PSS, and Nafion.

Further, example embodiments provide an electrochemical sensor for simultaneous detection of dopamine and serotonin, including the working electrode, and a kit and a neural electrode system for simultaneous detection of dopamine and serotonin, including the same.

Further, example embodiments provide the working electrode, and a method for manufacturing an electrochemical sensor for simultaneous detection of dopamine and serotonin including the same.

Further, example embodiments provide a method for simultaneous detection of dopamine and serotonin using an electrochemical sensor for simultaneous detection of dopamine and serotonin including the working electrode.

However, the technical problems to be achieved by the present disclosure are not limited to the above-mentioned problems, and other problems not mentioned will be clearly understood by those skilled in the art from the following description.

According to an aspect, in order to solve the foregoing problems, there is provided an electrochemical sensor for simultaneous detection of dopamine and serotonin including a working electrode containing: a reduced graphene oxide (rGO); poly(3,4-ethylenedioxythiophene):poly(4-styrenesulfonate) (PEDOT:PSS); and Nafion.

As one example embodiment of the present disclosure, the reduced graphene oxide may be one in which a graphene oxide deposited on a gold (Au) thin film is electrochemically reduced by cyclic voltammetry (CV), and may be preferably one in which it is repeatedly reduced 3 to 6 times at a scan rate of 50 mV/s in the voltage range of −1.5 to 0 V.

As another example embodiment of the present disclosure, the reduced graphene oxide may be one which is reduced at pH 3 to 5, preferably at pH 4 at room temperature and atmospheric pressure. Specifically, since the sensor according to the present disclosure has the lowest interfacial impedance, the highest charge storage capacity and the highest Raman spectroscopy result's ID/IG ratio, of rGO at pH 4, it has the most excellent reduction rate.

As another example embodiment of the present disclosure, the sensor may further include: a counter electrode including a platinum (Pt) wire or a gold thin film; and a reference electrode including a silver/silver chloride (Ag/AgCl) or gold thin film.

As another example embodiment of the present disclosure, the sensor may be one capable of simultaneously detecting dopamine and serotonin selectively by lowering the signal of an interfering substance having a negative charge, and the interfering substance may be any one or more selected from the group consisting of ascorbic acid (AA), uric acid (UA), glucose, epinephrine (EP), norepinephrine (NE), and combinations thereof, but is not limited thereto.

According to another aspect, there is provided a kit for simultaneous detection of dopamine and serotonin including the electrochemical sensor for simultaneous detection of dopamine and serotonin.

According to another aspect, there is provided a neural electrode system for simultaneous detection of dopamine and serotonin including the electrochemical sensor for simultaneous detection of dopamine and serotonin.

As one example embodiment of the present disclosure, the sensor may include a working electrode containing: a reduced graphene oxide (rGO); poly(3,4-ethylenedioxythiophene):poly(4-styrenesulfonate) (PEDOT:PSS); and Nafion, a counter electrode, and a reference electrode.

As another example embodiment of the present disclosure, the system may be one in which pluralities of counter electrodes and reference electrodes paired with a plurality of working electrodes are disposed in proximity.

As another example embodiment of the present disclosure, the system may be one in which single counter electrode and reference electrode that share a plurality of working electrodes are disposed to be spaced apart from each other.

According to another aspect, there is provided a method for manufacturing an electrochemical sensor for simultaneous detection of dopamine and serotonin, the method comprising the steps of: (1) performing electrophoretic deposition of a graphene oxide (GO) on a gold (Au) thin film working electrode; (2) preparing a reduced graphene oxide (rGO) by electrochemically reducing the graphene oxide deposited on the gold thin film; (3) preparing PEDOT:PSS by electropolymerizing a mixed solution of EDOT and PPS on the prepared reduced graphene oxide; and (4) coating the prepared PEDOT:PSS with Nafion to prepare a working electrode.

As one example embodiment of the present disclosure, the step (1) may be to selectively deposit an electrode material on a gold thin film working electrode in a sensor composed of a working electrode, a counter electrode, and a reference electrode, and such a deposition method means that the electrode material can be evenly formed on the entire gold thin film having a desired current applied thereto.

As another example embodiment of the present disclosure, the step (1) may be to perform electrophoretic deposition of a graphene oxide dispersed in water at 1 μA for 1,000 to 1,500 seconds, preferably 1,200 seconds.

As another example embodiment of the present disclosure, the gold thin film of the step (1) may have a thin thickness of 50 to 150 nm, preferably 100 nm.

As another example embodiment of the present disclosure, the step (2) may be to perform electrochemical reduction after drying a gold thin film having the graphene oxide deposited thereon at room temperature, preferably to perform drying at room temperature for 5 hours.

As another example embodiment of the present disclosure, the step (2) may be to electrochemically reduce the graphene oxide-deposited gold thin film by cyclic voltammetry (CV), preferably to perform reduction 3 to 6 times at a scan rate of 50 mV/s in a voltage range of −1.5 to 0 V.

As another example embodiment of the present disclosure, the step (2) may be to reduce the graphene oxide-deposited gold thin film at room temperature and normal pressure at pH 3 to 5, preferably at pH 4.

As another example embodiment of the present disclosure, the step (3) may be to wash the prepared reduced graphene oxide in deionized water (DI water), dry it at room temperature, and then conduct electropolymerization.

As another example embodiment of the present disclosure, the mixed solution of EDOT and PPS in the step (3) may be one having a concentration ratio of EDOT:PPS of 1:10, preferably one in which 0.01 M of EDOT and 0.1 M of PPS are mixed.

As another example embodiment of the present disclosure, the step (3) may be to electropolymerize the mixed solution of EDOT and PPS at 16 μA for 200 to 400 seconds, preferably 300 seconds.

As another example embodiment of the present disclosure, the step (4) may be to perform coating by diluting Nafion and dropping a small amount of diluted Nafion, at this time, the concentration of Nafion may be preferably 0.5% by weight.

As another example embodiment of the present disclosure, the sensor may further include: a counter electrode including a platinum (Pt) wire; and a reference electrode including silver/silver chloride (Ag/AgCl).

According to another aspect, there is provided a method for simultaneous detection of dopamine and serotonin, the method comprising the steps of: (1) treating a biological sample isolated from an individual on an electrochemical sensor for simultaneous detection of dopamine and serotonin according to the present disclosure; and (2) performing differential pulse voltammetry (DPV) on the sample-treated sensor at pH 6 to 8.

As one example embodiment of the present disclosure, the step (2) may be performed to a pulse amplitude of 0.05 V, a pulse period of 0.2 seconds, a pulse width of 0.05 seconds, and a step potential of 0.004 V.

Additional aspects of example embodiments will be set forth in part in the description which follows and, in part, will be apparent from the description, or may be learned by practice of the disclosure.

According to example embodiments, the electrode (rGO/PEDOT:PSS/Nafion) containing: a reduced graphene oxide; PEDOT:PSS; and Nafion, and the electrochemical sensor including the same are an electrochemical-based neurotransmitter detection chip, which is simple to prepare, requires a small amount of sample, and enables response signal detection as fast as within 7 seconds.

According to example embodiments, an rGO/PEDOT:PSS/Nafion electrode and the electrochemical sensor including the same have a very low detection limit for dopamine and serotonin detection since they have high conductivity and catalytic activity based on a large surface area and an excellent electron-conducting support material by containing an rGO having excellent biocompatibility, PEDOT:PSS as a conductive polymer, and negatively charged Nafion.

According to example embodiments, the rGO/PEDOT:PSS/Nafion electrode and the electrochemical sensor including the same enable selective high-sensitivity detection by reducing the signal interference of ascorbic acid (AA), uric acid (UA), glucose, epinephrine (EP), and norepinephrine (NE) having structures similar to dopamine and serotonin.

According to example embodiments, the rGO/PEDOT:PSS/Nafion electrode and the electrochemical sensor including the same can deposit it stably to a uniform thickness since they selectively deposit a graphene oxide (GO) on a thin Au electrode under a low current without high potential application or prepared GO suspension's drop-coating. In addition, since the deposited GO is electrochemically reduced using a harmless buffer solution at room temperature without using a reducing agent such as hydrazine, hydroxylamine, or hydroquinone, production is easy, fast, economical, and environmentally friendly.

According to example embodiments, since the rGO/PEDOT:PSS/Nafion electrode and the electrochemical sensor including the same show high reproducibility and long-term stability even in the detection of dopamine and serotonin in real human serum, they are applied to an ultra-small neural probe and provide simultaneous monitoring of multiple neurotransmitters including dopamine and serotonin so that they can be used in the diagnosis fields of degenerative brain diseases including Parkinson's disease, and mental disease, or can be used in basic brain function research, biological research and drug treatment using changes in dopamine and serotonin concentrations, and biochip application fields.

According to example embodiments, the effects of the rGO/PEDOT:PSS/Nafion electrode and the electrochemical sensor including the same are not limited to those mentioned above, and other effects not mentioned will be able to be clearly understood to those skilled in the art from the description below.

### BRIEF DESCRIPTION OF THE DRAWINGS

These and/or other aspects, features, and advantages of the invention will become apparent and more readily appreciated from the following description of example embodiments, taken in conjunction with the accompanying drawings of which:

FIG. 1 is a schematic diagram showing a method for manufacturing an electrochemical sensor for detection of dopamine and serotonin, including an rGO/PEDOT:PSS/Nafion electrode according to one example embodiment of the present disclosure;

FIGS. 2A and 2B show (a) EIS results and (b) CV curves (scan rate of 100 mV/s) of rGO electrodes in a 0.1 M PBS solution (pH 7.4) after electrochemical reduction of GO electrodes in different pH solutions (1.68, 4, 7.4, and 12), FIG. 2C shows results of comparing Raman spectra of rGO electrodes fabricated in solutions having different pH values, and FIG. 2D shows results of comparing the intensity ratios (I_{D}/I_{G}) of D and G bands obtained from Raman spectra of GO and rGO prepared using solutions having different pH values;

FIG. 3 is scanning electron microscope (SEM) images of the fabricated working electrodes, wherein all images are displayed at 10,000 times magnification of the electrode surface, the size bar indicates 5 μm, and the fabricated working electrodes are as follows: GO, rGO, rGO/PEDOT:PSS, and rGO/PEDOT:PSS/Nafion;

FIG. 4A shows FT-IR spectra of GO, rGO, PEDOT:PSS, rGO/PEDOT:PSS and rGO/PEDOT:PSS/Nafion electrodes deposited on an Au electrode, FIG. 4B shows an XPS distribution analysis result of the rGO/PEDOT:PSS/Nafion electrode, and FIGS. 4C and 4D show (c) EIS results and (d) CV curves (scan rate of 100 mV/s) of Au, GO, rGO, rGO/PEDOT:PSS and rGO/PEDOT:PSS/Nafion electrodes in a 0.1 M PBS solution (pH 7.4);

FIG. 5A shows DPV curves of Au, GO, rGO, rGO/PEDOT:PSS and rGO/PEDOT:PSS/Nafion electrodes containing 1 μM of serotonin, FIG. 5B shows CV curves of the rGO/PEDOT:PSS/Nafion electrode in 3 mM of serotonin at different scan rates of 10 to 100 mV/s, FIG. 5C shows a linear relationship (n=3) of the CV oxidation peak current of the serotonin concentration, and FIG. 5D shows an effect of pH (in a range of 5.0 to 9.0) on DPV (scan rate of 25 mV/s) of the rGO/PEDOT:PSS/Nafion electrode in 0.1 M PBS containing 1 μM of serotonin, wherein the relationship between pH and serotonin peak current (Iₚ, blue line) and peak potential (Eₚ, red line) is shown, and the error bars indicate the standard deviation;

FIG. 6 is a cross-sectional scanning electron microscope (SEM) image of rGO/PEDOT:PSS/Nafion, wherein the image is shown at 10,000 times magnification of the electrode surface, and the size bar indicates 2 μm;

FIGS. 7A and 7B show (a) DPV responses and (b) their calibration plot of the rGO/PEDOT:PSS/Nafion electrode for different concentrations of serotonin (0.05 to 50 μM), and FIGS. 7C and 7D show (c) DPV responses and (d) their calibration plot of the rGO/PEDOT:PSS/Nafion electrode for different concentrations of dopamine (0.5 to 75 μM);

FIGS. 8A and 8B show (A) DPV responses and (B) their calibration plot of the rGO/PEDOT:PSS/Nafion electrode for 1 μM of dopamine and different concentrations of serotonin (0.05 to 50 μM), and FIGS. 8C and 8D show (C) DPV responses and (D) their calibration plots of the rGO/PEDOT:PSS/Nafion electrode for simultaneously varying concentrations of serotonin and dopamine (0.1 to 50 μM) in a 0.1 M PBS (pH 7.4); and

FIG. 9A compares DPV responses of 1 μM of serotonin in the presence of 1 μM of serotonin alone and interfering substances (1,000 μM of AA, 50 μM of UA, 100 μM of glucose, 10 μM of epinephrine, and 10 μM of norepinephrine) treated with different concentrations (n=3), FIG. 9B compares DPV responses of six different sensors manufactured to confirm the reproducibility of the sensor according to one example embodiment of the present disclosure (n=3), FIG. 9C compares DPV responses for 5 weeks to confirm the long-term stability of the sensor according to one example embodiment of the present disclosure, and FIG. 9D compares DPV responses of the rGO/PEDOT:PSS/Nafion electrode for different concentrations of spiked serotonin in PBS buffer solution and serum (n=3).

### DETAILED DESCRIPTION

Since the electrochemical sensor including an rGO/PEDOT:PSS/Nafion electrode according to the present disclosure is manufactured by simply electropolymerizing PEDOT:PSS on a reduced graphene oxide (rGO) prepared at an optimal pH, and coating negatively charged Nafion thereon, it exhibits significantly improved catalytic properties and high conduction properties for the electroactivity of multiple neurotransmitters.

In order to confirm the dopamine and serotonin detection efficacy of the sensor, the present inventors carried out electrochemical characterization through electrochemical impedance spectroscopy (EIS), cyclic voltammetry (CV), and differential pulse voltammetry (DPV), and investigated the surface morphology, chemical state, and elemental composition of the electrode using a scanning electron microscope (SEM), Fourier-transform infrared (FT-IR) spectroscopy, Raman spectra, and high-resolution X-ray photoelectron spectroscopy (XPS).

As a result, it was confirmed that the electrochemical sensor including an rGO/PEDOT:PSS/Nafion electrode according to the present disclosure is capable of simultaneous high-sensitivity and selective detection of dopamine and serotonin by high affinity for cations.

Specifically, as a result of the electrochemical reaction, well-separated oxidation peaks were observed for mixtures containing various concentrations of dopamine and serotonin, and the dynamic sensing concentration ranges of dopamine and serotonin were shown to be 0.5 to 75 μM and 0.05 to 50 μM, respectively, and the detection limits were shown to be 0.5 and 0.05 μM, respectively. In the mixtures of dopamine and serotonin, the detection limits of dopamine and serotonin were 0.1 μM, and the sensitivities thereof were 99.3 and 86 μA/Mcm², respectively. Moreover, the electrochemical sensor according to the present disclosure exhibited high selectivity, reproducibility, stability, and recovery rate in the human serum spike test.

Accordingly, the present disclosure provides an electrochemical sensor for simultaneous detection of dopamine and serotonin, including; a working electrode including a reduced graphene oxide (rGO), PEDOT:PSS, and Nafion; a counter electrode including a platinum (Pt) wire; and a reference electrode including silver/silver chloride (Ag/AgCl).

In the present disclosure, the “working electrode” refers to an electrode in which a reaction of interest occurs in an electrochemical experimental system, may also be referred to as a cathode or anode depending on whether the reaction occurring at the electrode is an oxidation reaction or a reduction reaction, and may be substituted for a “working electrode” and used. Non-limiting examples of the material of the working electrode may be metals such as copper, platinum, silver, gold, palladium, ruthenium, rhodium, and iridium, semiconductors such as carbon, GaAs, CdS, and In₂O₃, or materials with surface treatment for each material, but may be preferably a gold electrode. In the present disclosure, electrophoretic deposition of GO was simply performed on a gold (Au) thin film, reduction was performed in an optimal pH buffer medium to form an rGO, then electropolymerization was performed with EDOT:PSS to form PEDOT:PSS, and PEDOT:PSS was coated with Nafion to prepare a working electrode in which rGO/PEDOT:PSS/Nafion was patterned.

“PEDOT:PSS” refers to poly(3,4-ethylenedioxythiophene):poly(4-styrenesulfonate) known as a conductive polymer.

“Nafion” is a material having ion exchange properties, and is a kind of sulfonated tetrafluoroethylene based fluoropolymer-copolymer.

In the present disclosure, the “reference electrode” refers to an electrode that provides a reference potential, and for example, a potential difference, i.e., a voltage, may be established between the reference electrode and the working electrode.

In the present disclosure, the “counter electrode” refers to an electrode in an electrochemical circuit that acts as a current source or sink to complete the electrochemical circuit, and may be substituted for “a control electrode” and “a counter electrode” and used.

Further, the present disclosure provides a method for simultaneously detecting dopamine and serotonin from a biological sample isolated from an individual using the electrochemical sensor.

In the present disclosure, the “individual” is not limited as long as it is a mammal such as a livestock, a human, or the like, but may be preferably a human.

In the present disclosure, the “biological sample” is an analysis target for determining whether dopamine and/or serotonin is contained or not, or measuring the concentration, and may include all biological samples such as whole blood, blood cells, serum, plasma, bone marrow fluid, sweat, urine, tears, saliva, skin, mucosa, and the like, and may be preferably serum isolated from humans.

Further, the present disclosure provides an information providing method for diagnosing a brain-nervous system disease or a mental disease based on the concentrations of dopamine and serotonin measured using the electrochemical sensor.

In the present disclosure, the “brain-nervous system disease” may be Parkinson's disease, Alzheimer's disease, Huntington's disease, amyotrophic lateral sclerosis, Creutzfeldt-Jakob disease, Pick disease, stroke, multiple system atrophy, dementia due to head trauma, vascular disease dementia, frontotemporal dementia (FTD), Corticobasal degeneration (CBD), Progressive supranuclear palsy (PSP), Lewy body dementia, tangle-predominant senile dementia, Argyrophilic grain disease, FTDP-17, Lytico-Bodig disease, learning disability, mild cognitive impairment, agnosia, forgetfulness, aphasia, apraxia, delirium, multiple sclerosis, brain cancer, traumatic brain injury (TBI), thrombosis, em-bolism, transient ischemic attack, lacune, cerebral apoplexy, cerebral infarction, cerebral circulatory metabolic disorder, brain function coma, or the like, but is not limited thereto.

In the present disclosure, the “mental disease” may be depression, lethargy, hyperactivity, attention deficit, autism, post-traumatic stress disorder (PTSD), anxiety disorder, sleep disorder, panic disorder, intellectual disability, memory loss, drug addiction, schizophrenia, obsessive compulsive disorder, megalomaniac, personality disorder, alcoholism, bipolar disorder, or the like, and may include physical symptoms resulting therefrom, for example, fatigue, indigestion, difficulty breathing, and the like, but is not limited thereto.

The terms used in the embodiments are used for the purpose of description only, and should not be construed as an intention to limit the present disclosure. The singular expression includes the plural expression unless the context clearly dictates otherwise. In the present specification, it should be understood that a term such as “comprise”, “have”, or the like is intended to designate that a feature, a number, a step, an operation, a component, a part, or a combination thereof described in the specification exists, but it does not preclude the possibility of existence or addition of one or more other features, numbers, steps, operations, components, parts, or combinations thereof.

Unless defined otherwise, all terms used herein, including technical or scientific terms, have the same meaning as those commonly understood by one of ordinary skill in the art to which the embodiments belong. Terms such as those defined in a commonly used dictionary should be interpreted as having a meaning consistent with the meaning in the context of the related art, and should not be interpreted in an ideal or excessively formal meaning unless explicitly defined in the present application.

The present disclosure can apply various transformations and can have various embodiments. Hereinafter, specific embodiments are illustrated in the drawings and described in detail in the detailed description. However, this is not intended to limit the present disclosure to specific embodiments, and should be understood to include all modifications, equivalents, and substitutes included in the spirit and technical scope of the present disclosure. In describing the present disclosure, if it is determined that a detailed description of a related known technology may obscure the gist of the present disclosure, the detailed description thereof will be omitted.

### Experimental Example 1. Chemicals and Reagents

To fabricate the flexible sensor configuration, polyimide (PI, VTEC 1388) was obtained from Richard Blaine International, Inc., Philadelphia, PA, USA. DNR-L300-30 was obtained from Dongjin, Seoul, Korea. AZ 9260 was obtained from AZ Electronic Materials, NJ, USA. Phosphate buffer saline (0.1 M PBS, pH 7.4) was obtained from Duksan General Science in Korea.

For the electrochemical reduction, the phosphate buffer solution containing K₂HPO₄ and KH₂PO₄ was adjusted to the preferred pH. Dopamine (DA), serotonin (5-HT), ascorbic acid (AA), uric acid (UA), glucose, epinephrine (EP), and norepinephrine (NE) were purchased from Sigma-Aldrich for electrochemical analysis. Commercially-sterilized and filtered human serum (derived from human male AB plasma, USA origin, code H4522) was obtained from Sigma-Aldrich for spike testing.

### Experimental Example 2. Fabrication of Flexible Sensor

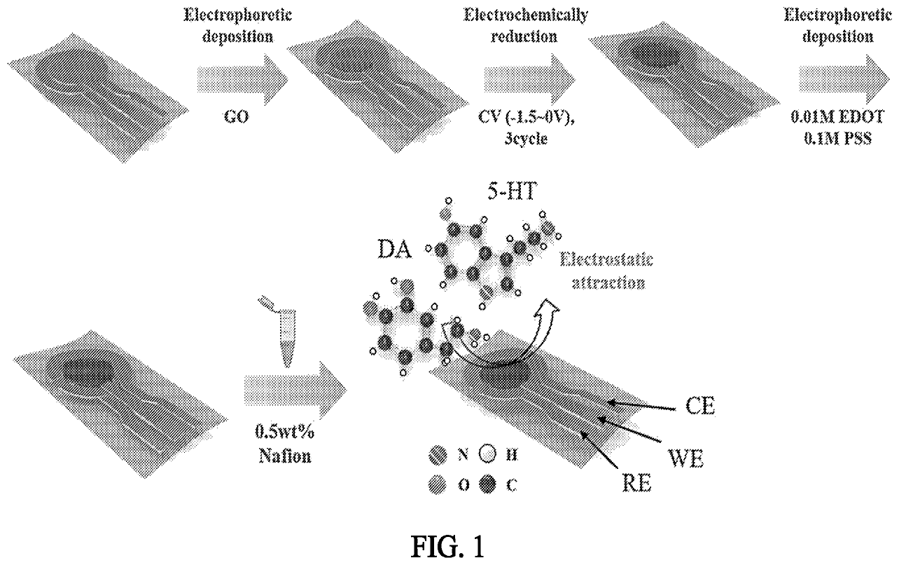

FIG. 1 shows a process of fabricating a flexible sensor, and includes a working electrode, a counter electrode, and a reference electrode. A first polyimide (PI, thickness of m) was spin-coated as a substrate layer. After curing, a negative photoresist was spin-coated on the polyimide layer for a lift-off process. After patterning, Cr/Au (10/100 nm) was deposited using an electron beam evaporator. After the lift-off process, a second polyimide was spin-coated and cured into an insulating layer (thickness of 3 μm). The second polyimide layer was coated with a positive photoresist to open an electrode site and a connector pad. After patterning, the exposed polyimide pattern was etched by reactive ion etching. The flexible sensor was easily separated from the wafer using a laser dicing machine in order to cut the perimeter of the sensor.

### Experimental Example 3. Manufacturing of Flexible Sensor Including rGO/PEDOT:PSS/Nafion Electrode

As shown in FIG. 1, a method for manufacturing a flexible sensor including rGO/PEDOT:PSS/Nafion on a thin Au working electrode is as follows. First, GO was electrophoretically deposited at 1 μA for 1,200 seconds. Thereafter, the working electrode containing GO selectively deposited on thin Au was dried at room temperature for 5 hours. Second, in order to perform the electrochemical reduction of the deposited GO, Au on which GO was deposited as the working electrode, a Pt wire as the counter electrode, and Ag/AgCI as the reference electrode were used. To determine the optimal rGO conditions, electrochemical reduction according to cyclic voltammetry was performed in buffer solutions with various pH values (e.g., 1.68, 4, 7.4, and 12). At this time, a potential window was applied from −1.5 V to 0 V, and the scan rate was performed three times at 50 mV/s. After electrochemical reduction, the color of the GO-coated electrode changed from brown to black, and rGO was washed three times in deionized (DI) water and dried at room temperature. Third, rGO was treated with the prepared EDOT:PSS solutions (0.01 M EDOT and 0.1 M PSS in deionized water) by electropolymerization (at 16 μA for 300 seconds). Finally, rGO/PEDOT:PSS was coated with 0.5% by weight of Nafion (1 μl) to fabricate an rGO/PEDOT:PSS/Nafion electrode according to one embodiment of the present disclosure.

### Experimental Example 4. Electrochemical Characterization

The electrochemical performance of the sensor according to one embodiment of the present disclosure was evaluated at room temperature by Autolab (PGSTAT 302N, NOVA software, Ecochemie, Utrecht, The Netherlands). Cyclic voltammetry (CV), electrochemical impedance spectroscopy (EIS), and differential pulse voltammetry (DPV) were performed using an Au reference electrode, an Au counter electrode, an Au (diameter of 2.4 mm) working electrode, and prepared GO-based working electrodes (GO, rGO, rGO/PEDOT:PSS, and rGO/PEDOT:PSS/Nafion).

CV with a potential limit of −0.2 to 0.8 V was performed at a scan rate of 100 mV/s, and the frequency range of EIS was 1 to 105 Hz. The parameters of the DPV measurement were set as follows: a scan rate of 25 mV/s, a pulse width of 0.06 seconds, and an amplitude of 30 mV. To avoid spontaneous oxidation of dopamine and serotonin, fresh solutions were prepared daily and stored in a dark room at 4° C. All experiments were performed at ambient temperature. The selectivity of the rGO/PEDOT:PSS/Nafion electrode was investigated via the DPV oxidation current response to serotonin (1 μM). At this time, ascorbic acid (AA, 1,000 μM), uric acid (UA, 50 μM), glucose (100 μM), epinephrine (EP, 10 μM), and norepinephrine (NE, 10 μM) was tested in a 0.1 M PBS (pH 7.4). The reproducibility of rGO/PEDOT:PSS/Nafion was investigated through six electrodes and serotonin oxidation peak current. At this time, in order to evaluate the reproducibility and selectivity of serotonin detection in the 0.1 M PBS (pH 7.4), DPV was calculated with 1 μM of serotonin (n=3).

### Experimental Example 5. Surface Morphology and Elemental Analysis

The surface morphology and elemental analysis of the electrodes were evaluated with a scanning electron microscope (SEM, Regulus 8230). Fourier transform infrared (FT-IR) spectra were captured using a Thermo Nicolet iS10 spectrometer and pelleted in KBr for FT-IR analysis. Raman spectra were recorded using a Renishaw Raman microscope with a neodymium-doped yttrium aluminum garnet laser containing a photon beam of a wavelength of 532 nm. X-ray photoelectron spectroscopy (XPS, Ulvac, Japan) was irradiated with a monochromatic AI Kα, X-ray source. XPS distribution analysis (XPS depth profiling) was performed in order to obtain the approximate thickness of the layer. A high-energy Ar⁺ ion beam having an acceleration voltage of 2 kV was used to sputter samples layer by layer on the top surface within an area of 1×1 mm². After each successive sputter cycle, Au4f, C1s, F1s, O1s and S2p peak areas were registered and analyzed.

### Experimental Example 6. Serum Spiking Test

For practical validation of the sensor including the rGO/PEDOT:PSS/Nafion electrode for simultaneous detection of dopamine and serotonin, all serum samples were prepared without further processing or dilution. Various concentrations of dopamine and serotonin were tested in spiked serum samples. For quantitative analysis, the DPV current response of the sensor including the rGO/PEDOT:PSS/Nafion electrode was checked and compared with standard serotonin solution and serotonin spike serum through DPV. Current responses measured from spiked serum samples and standard solution that have various dopamine and serotonin concentrations were calculated and compared using recovery rate (% recovery rate=Cᵢ/Cₒ/Cₓ). At this time, Cᵢ and Cₒ are the serotonin concentrations experimentally obtained in the spike and blank serum samples, respectively, and Cₓ is the concentration of serotonin actually spiked in the serum samples.

### Experimental Results 1. Characterization of Reduced GO Electrode

Although GO can be electrochemically reduced in a wide pH range of 1.5 to 12.5, optimal pH conditions for the medium are essential to ensure that the rGO surface has excellent electrocatalytic properties. Reduced GO films were prepared through cyclic voltammetry for a potential range of −1.5 to 0 V in buffer solutions of various pH conditions.

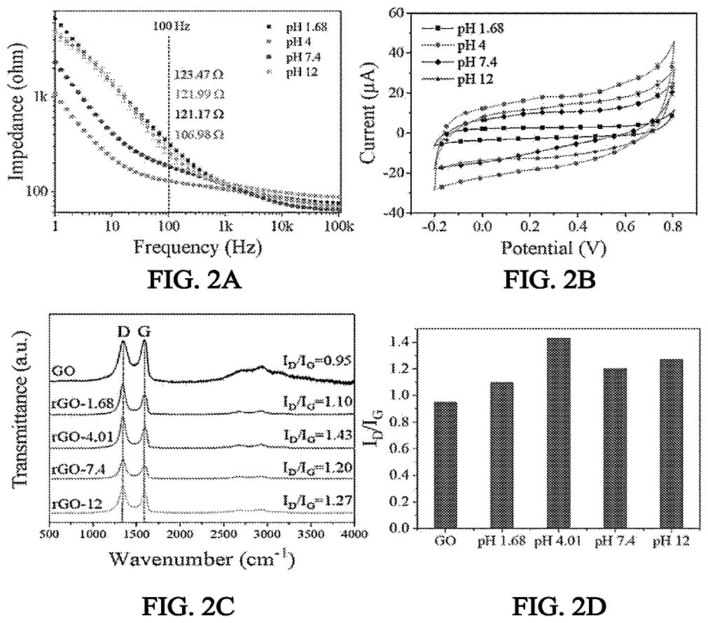

FIGS. 2A and 2B show interfacial impedances and CV curves of rGO electrodes fabricated under various pH conditions in a 0.1 M PBS solution. Electrochemical reduction was achieved by restoring the graphene domain of carbon bonds by removing the oxygen functional groups of GO. Therefore, the concentration of H⁺ in the medium strongly affects the performance of rGO. As shown in FIGS. 2A and 2B, the interfacial impedances of rGO fabricated at pH 1.68, 4.01, 7.4, and 12 were 123.47±9.75, 106.98 1.13, 121.17±0.38, and 121.99±5.14Ω at 100 Hz, respectively. Charge storage capacity (CSC), such as actual activation area, increased rapidly at pH 4 and decreased at other pH conditions (0.24, 1.33, 0.79, and 1.07 mC/cm²). In such a pH range (pH<2; pH>10), electrochemical reduction exhibited a side reaction, which may cause reduction competition with H⁺ and formation of hydrogen bubbles in the working electrode. Therefore, the medium of the neural region provides favorable conditions for the reduction of GO without an interfering side reaction. That is, it was confirmed that pH 4.01 was most suitable for electrochemical reduction.

As a result of Raman spectroscopy measurement for quantitative analysis of rGO in FIG. 2C, the characterized D and G bands showed peaks of about 1,345 cm⁻¹ and 1,590 cm⁻¹, respectively. At this time, the D and G bands correspond to the sp2 and sp3 carbon stretching modes. The intensity ratio (I_{D}/I_{G}) of the D and G bands was a measure of the size of the sp2 ring in the sp2 and sp3 carbon binding domains, and this means that GO was successfully reduced. FIG. 2D shows that the I_{D}/I_{G} ratio of GO significantly increases after electrochemical reduction. The I_{D}/I_{G} ratios of rGO (1.10, 1.43, 1.20, and 1.27, respectively) at pH 1.68, 4.01, 7.4, and 12 were higher than those of GO, and this means a smaller increase in the number of sp2 domains after electrochemical reduction. After reduction at pH 4.01, the intensity ratio (e.g., I_{D}/I_{G}) results from a decrease in defect density according to a rapid electrochemical reduction rate. These results support the fact that the electrochemical characterization properties shown in FIGS. 2A and 2B, specifically the concentrations of H⁺, have a great effect on the electrochemical reduction, resulting in improved performance of rGO. Therefore, the pH 4.01 condition was selected as the buffering condition for rGO.

### Experimental Results 2. Characterization of rGO/PEDOT:PSS/Nafion Electrode

### 2.1. Morphological Analysis

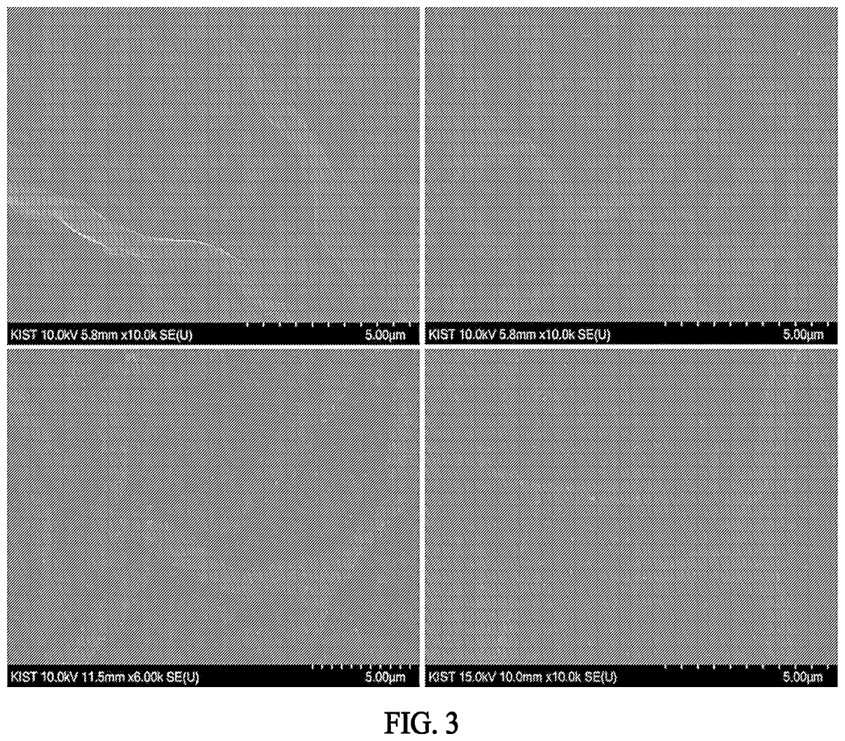

FIG. 3 is one in which the surface morphologies of the fabricated GO, rGO, rGO/PEDOT:PSS and rGO/PEDOT:PSS/Nafion electrodes are measured and compared by SEM. GO(a) and rGO(b) electrodes deposited on thin Au electrodes exhibited random wrinkle structures. The surfaces of the rGO/PEDOT:PSS(c) and rGO/PEDOT:PSS/Nafion(d) electrodes showed clearly different structures compared to the structure of rGO, and this is thought to be because the well-distributed PEDOT:PSS covers the ridge formed by rGO. In addition, the surface of the rGO/PEDOT:PSS/Nafion electrode is shown to be similar to that of the rGO/PEDOT:PSS electrode, and this suggests that the well-distributed thin Nafion layer does not affect the structure of the rGO/PEDOT:PSS electrode.

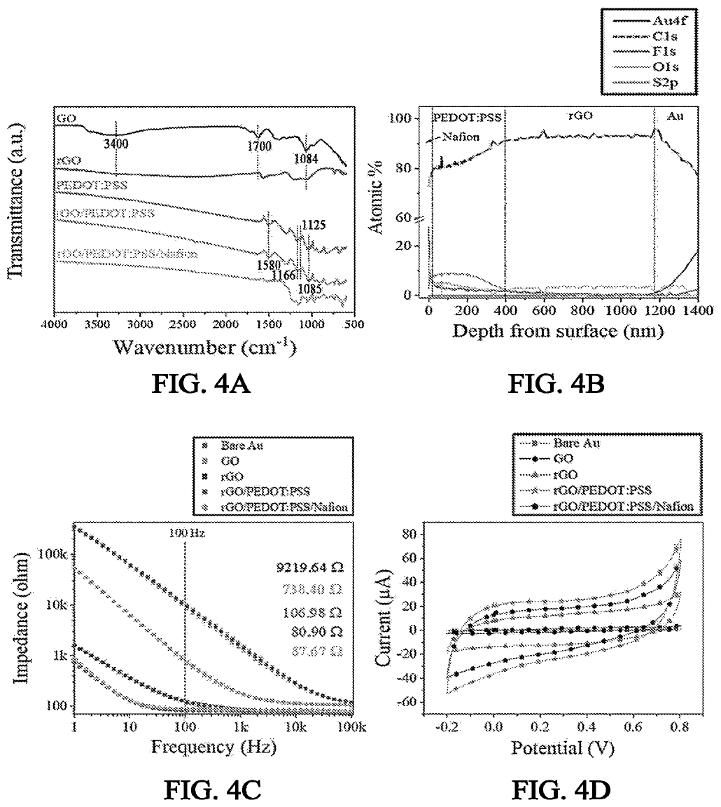

Chemical transformation was confirmed by irradiating the fabricated GO, rGO, rGO/PEDOT:PSS, and rGO/PEDOT:PSS/Nafion electrodes with FT-IR. FIG. 4A shows the removal of oxygenated functional groups in rGO compared to GO. After electrochemical reduction, the transmittance disappeared clearly at 3,400 cm⁻¹, 1,700 cm⁻¹, and 1,084 cm⁻¹, and this means that the oxygenated groups were essentially removed. The above results suggest that most oxygen oxygenated in GO can be effectively removed by electrochemical reduction. The transmittance curve of the rGO/PEDOT:PSS electrode reflected the formation of new bonds by PEDOT:PSS. Specifically, vibrations at 1,580 cm⁻¹ and 1,508 cm⁻¹ (thiophene ring) and 1,166 cm⁻¹ and 1,125 cm⁻¹ (phenyl group) were confirmed in the spectra of the rGO/PEDOT:PSS electrode. This indicates that PEDOT:PSS was well configured on the rGO surface by electropolymerization. Interestingly, the peak intensity of the rGO/PEDOT:PSS/Nafion electrode at 1,500 to 1,000 cm⁻¹ increased significantly after coating of the Nafion layer, and this is because of the bonding of symmetric C-F stretching at 1,145 cm⁻¹ and asymmetric C-F stretching at 1,201 cm⁻¹.

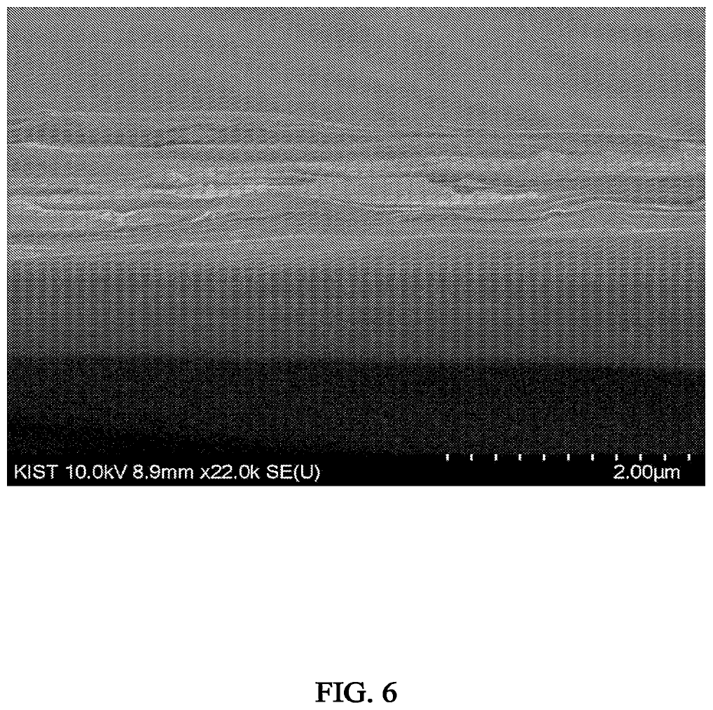

To investigate the presence of different material layers in the rGO/PEDOT:PSS/Nafion electrode, XPS distribution analysis was performed for Au4f, C1s, F1s, O1s and S2p level regions. FIG. 4A shows the XPS distribution analysis results, and it is possible to provide the approximate thickness of each material layer for the rGO/PEDOT:PSS/Nafion electrode through increase or decrease in each of the measured atomic concentration and intensity. As a result, the thicknesses of Nafion, PEDOT:PSS, and rGO were about 3,400 and 800 nm, respectively. The proportion of atomic carbon decreased sharply at the surface between rGO and Au. Such results agree relatively well with the cross-sectional SEM image of the rGO/PEDOT:PSS/Nafion electrode shown in FIG. 6.

### 2.2. Electrochemical Properties

To investigate the electrochemical performance of the fabricated working electrode, interfacial impedances and cyclic voltammetry of Au, GO, rGO, rGO/PEDOT:PSS, and rGO/PEDOT:PSS/Nafion electrodes were measured in a 0.1 M PBS solution (pH 7.4), and the results are shown in FIGS. 4C and 4D. The interfacial impedances of the Au, GO, rGO, rGO/PEDOT:PSS, and rGO/PEDOT:PSS/Nafion electrodes measured at 100 Hz were 9,219.64±72.63, 738.40 7.4, 360.11±7.12, 80.90 0.7, and 87.67±0.57Ω, respectively. The interfacial impedance of the rGO/PEDOT:PSS electrode was extremely improved compared to other electrodes due to its excellent conductivity and low charge transfer resistance.

PEDOT:PSS exhibited an extended current response indicating an increased number of electrochemically active sites. In the measured CV curves, the CSC values implying the accumulated charges gradually expanded in the order of Au<GO<rGO<rGO/PEDOT:PSS<rGO/PEDOT:PSS/Nafion, and exhibited values of 0.15, 1.14, 13.3, 22.6, and 15.6 mC/cm², respectively. The rGO/PEDOT:PSS electrode was strengthened to have higher capacitance values after electrochemical reduction and deposition.

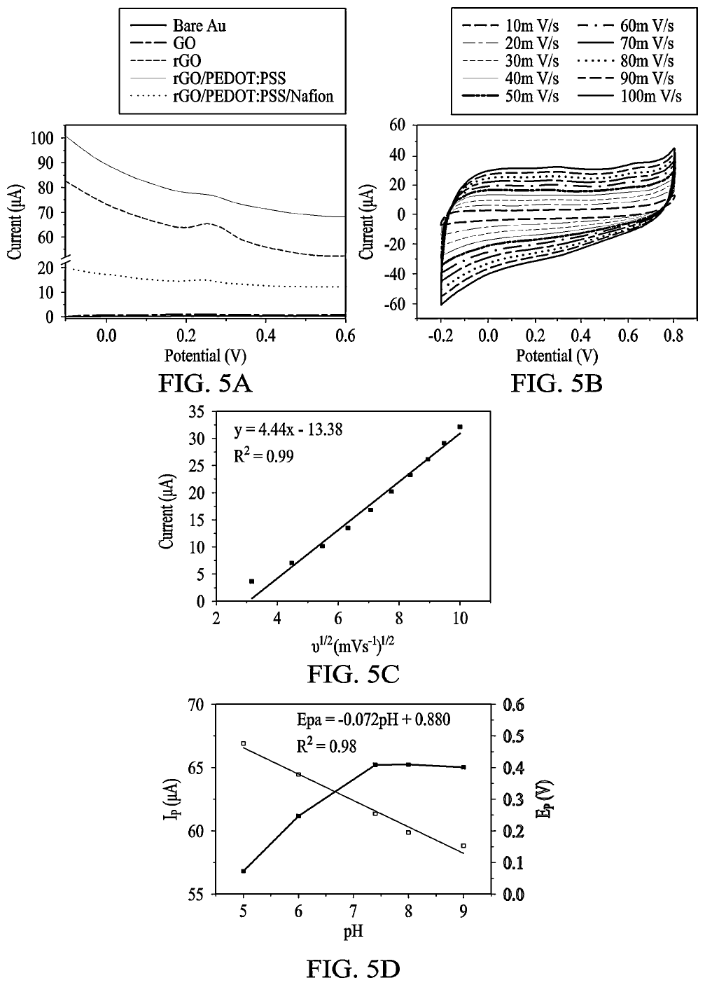

As shown in FIG. 5A, in the DPV response curves of sensors including Au, GO, rGO, rGO/PEDOT:PSS and rGO/PEDOT:PSS/Nafion electrodes for 1 μM of serotonin, the peak responses of the sensors without Au and GO were difficult to discern, whereas the oxidation peak responses of the sensors having rGO and rGO/PEDOT:PSS were slightly improved. In the case of the rGO/PEDOT:PSS/Nafion sensor, the oxidation peak of serotonin was clearly observed compared to the Au, rGO, and rGO/PEDOT:PSS sensors. This may be caused by the properties of Nafion to be negatively charged with respect to strongly attracted cations such as serotonin.

### Experimental Results 3. Effects of Scan Rate and pH

The effects of the scan rates evaluated using the CV curves were shown in FIGS. 5B and 5C. In FIG. 5B, the CV curves of the rGO/PEDOT:PSS/Nafion electrode were recorded in PBS (pH 7.4) at a scan rate of 10 to 100 mV/s. Therefore, a good linear relationship (R²=0.98) between the oxidation peak current and the square root of the scan rate was shown in FIG. 5C. This means that a typical diffusion-controlled electron transfer process occurred in the rGO/PEDOT:PSS/Nafion electrode.

The effect of pH on the oxidation of serotonin in a sensor including the rGO/PEDOT:PSS/Nafion electrode was investigated with DPV in the pH range of 5 to 9. FIG. 5D shows the relationship between pH and peak current during oxidation of serotonin. That is, it can be seen that when the pH increases, the oxidation potential shifts negatively so that the highest peak current can be obtained at pH 7.4. Therefore, the maximum value of the current at pH 7.4 suggests a high electrochemical activity of serotonin for the rGO/PEDOT:PSS/Nafion electrode. The calculated regression equation is Eₚₐ (V)=−0.072 pH+0.88 (R²=0.98). The slope is −72 mV/pH, which is close to the theoretical value of −59 mV/pH. Through this, it can be confirmed that two electrons and two protons were transferred during the oxidation process of serotonin as shown in the Nernst equation. Therefore, pH 7.4 was chosen as an appropriate value for further studies on serotonin detection in rGO/PEDOT:PSS/Nafion.

### Experimental Results 4. DPV Responses of rGO/PEDOT:PSS/Nafion Electrode to Dopamine and Serotonin

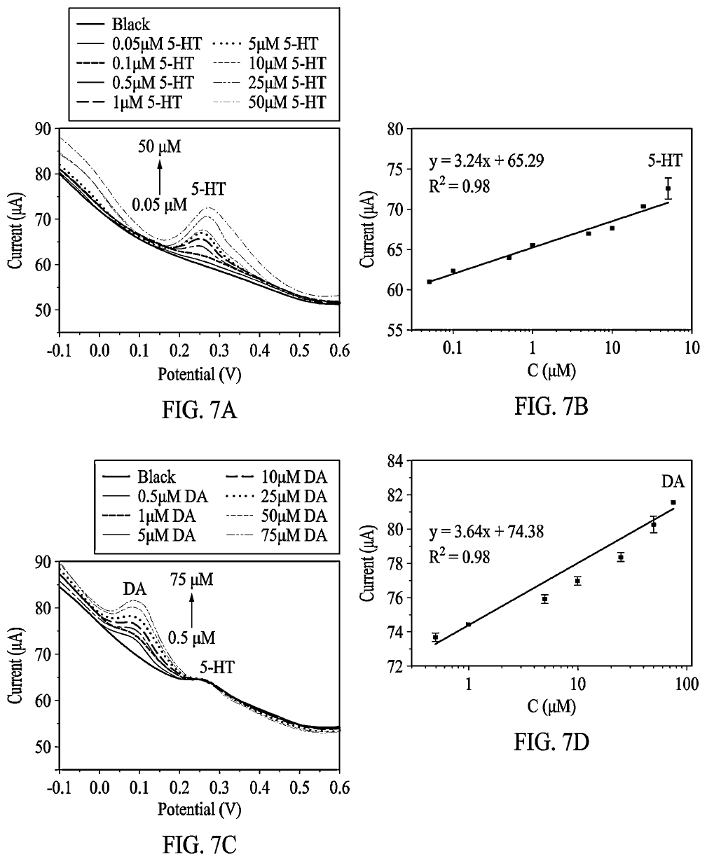

As shown in FIG. 7A, serotonin detection of a sensor including an rGO/PEDOT:PSS/Nafion electrode at various concentrations (0.05 to 50 μM) using the DPV technique in a 0.1M PBS (pH 7.4) was tested. The oxidation peak current of the sensor including the rGO/PEDOT:PSS/Nafion electrode gradually increased by about 0.25 V as the concentration of serotonin increased from 0.05 to 50 μM. The linearity of the sensor's peak current (IP) with respect to the concentration of serotonin is shown in FIG. 7A. The linear regression equation for serotonin is IP (μA)=3.24 C (μM)+65.29 (R²=0.98). The sensitivity and limit of detection (LOD) of the sensor including the rGO/PEDOT:PSS/Nafion electrode were 71.66 μA μM⁻¹ cm⁻² and 0.05 μM, respectively.

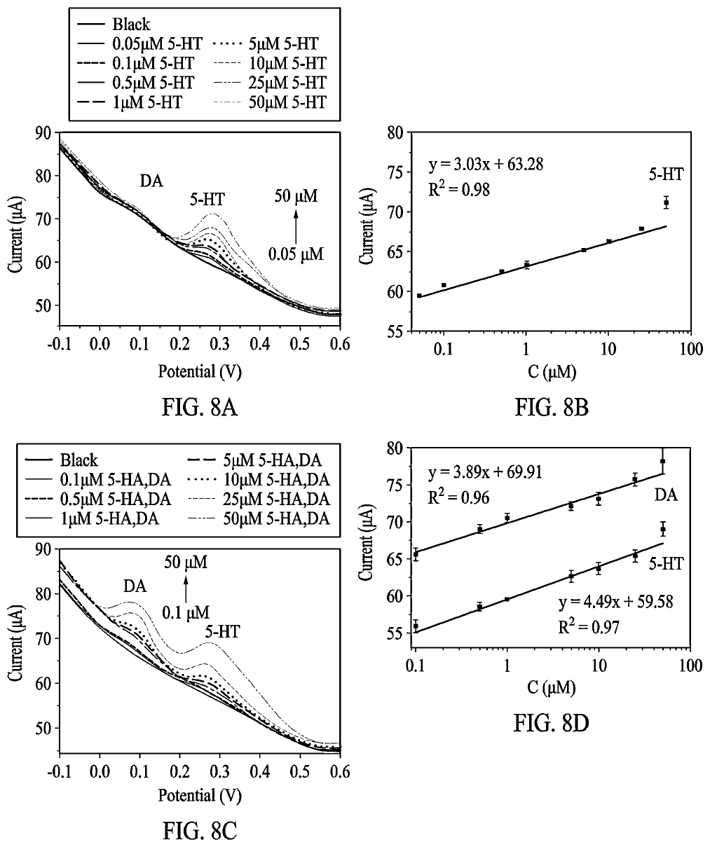

To confirm the selective detection of dopamine and serotonin, the concentration of one species was fixed and the concentration of the other species was changed. Simultaneous detection of two analytes was performed by simultaneously changing the concentration of one species in the prepared mixture. As shown in FIGS. 7 and 8, the current responses to dopamine and serotonin were shown in separate potential regions (0.09 V and 0.25 V) having excellent linearity. At this time, the sensitivities to dopamine and serotonin were 80.5 and 67.1 μA M-cm⁻², respectively. The concentration of dopamine was changed to 0.5 to 75 μM in a state that the concentration of serotonin was fixed to 1 μM, and the concentration of serotonin was changed to 0.5 to 75 μM in a state that the concentration of dopamine was fixed to 1 μM. As a result, the limit of detection of dopamine was shown to be 0.5 μM (FIG. 7C), the limit of detection of serotonin was shown to be 0.05 μM (FIG. 7D), and these are numerical values sufficient for practical use. In FIG. 8C, the sensor including the rGO/PEDOT:PSS/Nafion electrode exhibited linear current responses with increasing the concentrations of dopamine and serotonin. In addition, the correlation linear responses show only small changes (FIGS. 7D, 8B, and 8D), indicating that the sensor according to the present disclosure is excellent in resistance to interference and selectivity. At this time, the sensitivities of the sensor for detection of dopamine and serotonin including the rGO/PEDOT:PSS/Nafion electrode were 99.3 and 86 μA/μMcm², respectively.

Compared with previous studies, the sensor according to the present disclosure exhibited excellent distinctiveness in the simultaneous response to dopamine and serotonin. The improved performance of the present disclosure may be due to the high catalytically active site of rGO/PEDOT:PSS/Nafion based on the surface, large surface area, and excellent electron-conducting support material of negatively charged Nafion.

### Experimental Results 5. Selectivity

The presence of interference is an important parameter determining the selectivity of the rGO/PEDOT:PSS/Nafion electrode for serotonin detection. The coexistence of serotonin and other interfering species may induce mixed response currents due to their close oxidation potential.

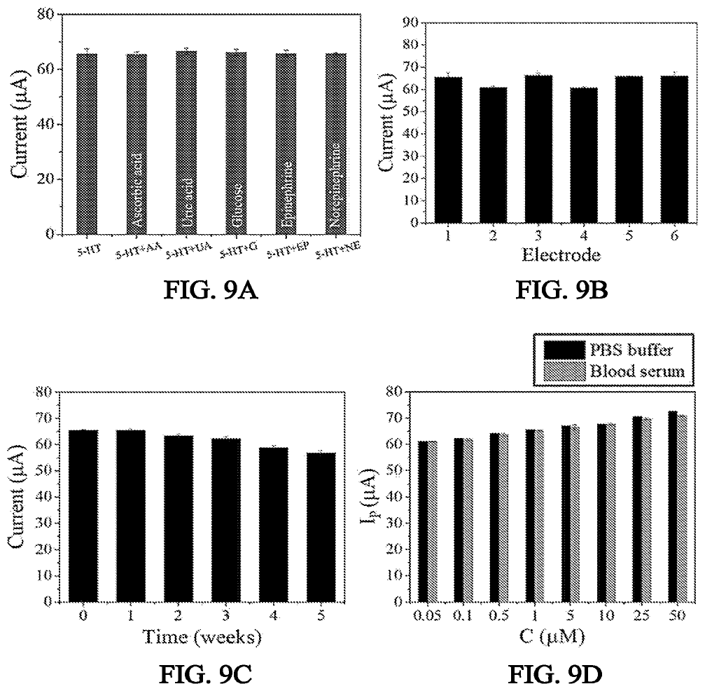

Therefore, the selectivities of the sensor including the rGO/PEDOT:PSS/Nafion electrode were investigated with DPV oxidation current responses of serotonin (1 μM). FIG. 9A and Table 1 show results of testing ascorbic acid (AA, 1,000 μM), uric acid (UA, 50 μM), glucose (100 μM), epinephrine (EP, 10 μM), and norepinephrine (NE, 10 μM) in a 0.1 M PBS (pH 7.4).

TABLE 1

| | Concentration | | | |
|---|---|---|---|---|
| | of interfering | Concentration | | |
| Interfering | substance | of serotonin | Serotonin DPV's | Peak current of |
| substance added | (μM) | (μM) | potential (V) | serotonin (μA) |
| Ascorbic acid (AA) | 1000 | 1 | 0.250 | 65.72 |
| Uric acid (UA) | 50 | 1 | 0.247 | 65.95 |
| Glucose | 100 | 1 | 0.251 | 65.85 |
| Epinephrine (EP) | 10 | 1 | 0.248 | 65.73 |
| Norepinephrine | 10 | 1 | 0.252 | 65.86 |
| (NE) | | | | |

According to Table 1, the peak oxidation currents of serotonin were maintained at 65 μA, and it was found that even if interfering substances with higher concentrations were present, the responses of serotonin DPV were not significantly interfered. Particularly, the oxidation potential of serotonin at 1,000 μM of ascorbic acid did not shift at 0.25 V due to repulsion from the negatively charged Nafion surface. Based on the above results, it was confirmed that the sensor including the rGO/PEDOT:PSS/Nafion electrode according to the present disclosure has higher anti-interference abilities for ascorbic acid, uric acid, glucose, epinephrine, and norepinephrine in the detection of serotonin. This means the excellent selectivity of the present disclosure for serotonin.

### Experimental Results 6. Reproducibility and Stability of Sensor Including rGO/PEDOT:PSS/Nafion Electrode

As shown in FIG. 9B, the reproducibility of rGO/PEDOT:PSS/Nafion was investigated using the six fabricated electrodes. Specifically, the oxidation peak currents of serotonin were measured through DPV in a 0.1M PBS (pH 7.4). As a result, the standard deviation was so small that the standard deviation was only 7.84%, and this means that the sensor according to the present disclosure has high reproducibility and is suitable for mass production application fields. Long-term stability for rGO/PEDOT:PSS/Nafion over a 5-week time interval was studied (FIG. 9C). After 5 weeks, the peak current of serotonin changed from 65 μA to 56.6 μA, and at this time, the deviation of the oxidation peak current of serotonin was 13%. The above results suggest that the sensor according to the present disclosure exhibits excellent reproducibility, repeatability, and stability as a sensor for serotonin detection.

### Experimental Results 7. Analysis of Real Serum Samples

In order to demonstrate that the sensor including the rGO/PEDOT:PSS/Nafion electrode according to the present disclosure can be used for clinical detection, a sensor including the rGO/PEDOT:PSS/Nafion electrode was tested, and spiked real serum samples were used to compare recovery rates.

The recovery rates were 93.3% to 101.8% (RSD, n=3) based on the detection of serotonin in human serum (Table 2), and the sensor including the rGO/PEDOT:PSS/Nafion electrode effectively detected serotonin from human serum samples.

TABLE 2

| Sample | Spiked (μM) | Found (μM) | Recovery (%) | RSD (%)(n = 3) |
|---|---|---|---|---|
| Serum 1 | 0.05 | 0.0485 | 96.9 | 2.2 |
| Serum 2 | 0.1 | 0.0972 | 97.2 | 2.1 |
| Serum 3 | 0.5 | 0.4784 | 95.6 | 4.3 |
| Serum 4 | 1 | 0.9615 | 96.2 | 6.5 |
| Serum 5 | 5 | 4.9124 | 98.2 | 5.4 |
| Serum 6 | 10 | 9.3325 | 93.3 | 5.9 |
| Serum 7 | 25 | 24.9232 | 99.6 | 4.8 |
| Serum 8 | 50 | 50.9085 | 101.8 | 6.6 |

Interestingly, the above results showed excellent recovery rates for 0.05 to 5 μM, indicating that the sensor according to the present disclosure can efficiently detect even low levels of serotonin in human serum, and reproducibility and feasibility are also very high. In addition, in order to investigate the detection of serotonin by the sensor including the rGO/PEDOT:PSS/Nafion electrode in the presence of dopamine, dopamine and serotonin (0.1 to 5 μM) were added to serum samples (Table 3).

TABLE 3

| | | | | | | | RSD (%) | |
|---|---|---|---|---|---|---|---|---|
| | Spiked (μM) | | Found (μM) | | Recovery (%) | | (n = 3) | |
| Sample | DA | 5-HT | DA | 5-HT | DA | 5-HT | DA | 5-HT |
| Serum 1 | 0.1 | 0.1 | 0.0933 | 0.0961 | 93.3 | 96.1 | 2.3 | 2.5 |
| Serum 2 | 0.5 | 0.5 | 0.4602 | 0.4456 | 92.4 | 89.1 | 2.4 | 3.3 |
| Serum 3 | 1 | 1 | 0.9188 | 0.9652 | 91.9 | 96.5 | 3.5 | 2.7 |
| Serum 4 | 5 | 5 | 4.4708 | 4.7467 | 89.4 | 94.9 | 4.0 | 4.9 |

The recovery rates of dopamine and serotonin were 89.4 to 93.3% and 89.1 to 96.1%, respectively, and the relative standard deviations (RSD) of three replicates were less than 5%. Therefore, the sensor including the rGO/PEDOT:PSS/Nafion electrode showed an excellent recovery range in undiluted serum samples, and this suggests that the present disclosure is suitable for detection of dopamine and serotonin in actual sample analysis.

Although Examples have been described with reference to the limited drawings as described above, those skilled in the art may apply various technical modifications and variations based on the above description. For example, although the described techniques are performed in an order different from the described method, and/or the constituent elements such as the described system, structure, apparatus, circuit, etc. are coupled or combined in a form different from the described method, or replaced or substituted by other constituent elements or equivalents, appropriate results may be accomplished.

Therefore, other embodiments, other Examples, and equivalents to the patent claim scope also belong to the scope of the claims to be described later.

What is claimed is:

1. An electrochemical sensor for simultaneous detection of dopamine and serotonin comprising a working electrode containing:
a reduced graphene oxide (rGO);
poly(3,4-ethylenedioxythiophene):poly(4-styrenesulfonate) (PEDOT:PSS); and
Nafion.

2. The electrochemical sensor of claim 1, wherein the reduced graphene oxide is one in which a graphene oxide deposited on a gold (Au) thin film is electrochemically reduced.

3. The electrochemical sensor of claim 1, wherein the reduced graphene oxide is one which is reduced at pH 3 to 5.

4. The electrochemical sensor of claim 1, wherein the reduced graphene oxide is one which is reduced through the application of cyclic voltammetry (CV) in a voltage range of −1.5 to 0 V.

5. The electrochemical sensor of claim 1, wherein the sensor further comprises:
a counter electrode including a platinum (Pt) wire or a gold thin film; and
a reference electrode including a silver/silver chloride (Ag/AgCl) or gold thin film.

6. The electrochemical sensor of claim 1, wherein the sensor is one capable of simultaneously detecting dopamine and serotonin selectively by lowering the signal of an interfering substance having a negative charge.

7. The electrochemical sensor of claim 6, wherein the interfering substance is any one or more selected from the group consisting of ascorbic acid (AA), uric acid (UA), glucose, epinephrine (EP), norepinephrine (NE), and combinations thereof.

8. A kit for simultaneous detection of dopamine and serotonin, the kit comprising the sensor of claim 1.

9. A neural electrode system for simultaneous detection of dopamine and serotonin, the neural electrode system comprising the sensor of claim 5.

10. The neural electrode system of claim 9, wherein the system is one in which pluralities of counter electrodes and reference electrodes paired with a plurality of working electrodes are disposed in proximity.

11. The neural electrode system of claim 9, wherein the system is one in which single counter electrode and reference electrode that share a plurality of working electrodes are disposed to be spaced apart from each other.

12. A method for manufacturing an electrochemical sensor for simultaneous detection of dopamine and serotonin, the method comprising the following steps of:
(1) performing electrophoretic deposition of a graphene oxide (GO) on a gold (Au) thin film working electrode;
(2) preparing a reduced graphene oxide (rGO) by electrochemically reducing the graphene oxide deposited on the gold thin film;
(3) preparing PEDOT:PSS by electropolymerizing a mixed solution of EDOT and PPS on the prepared reduced graphene oxide; and
(4) coating the prepared PEDOT:PSS with Nafion to prepare a working electrode.

13. The method of claim 12, wherein the step (2) is to reduce the graphene oxide-deposited gold thin film at pH 3 to 5.

14. The method of claim 12, wherein the step (2) is to reduce the graphene oxide-deposited gold thin film by cyclic voltammetry (CV) in a voltage range of −1.5 to 0 V.

15. The method of claim 12, wherein the mixed solution of EDOT and PPS in the step (3) is one having a concentration ratio of EDOT:PPS of 1:10.

16. The method of claim 12, wherein the sensor further comprises:
a counter electrode including a platinum (Pt) wire; and
a reference electrode including silver/silver chloride (Ag/AgCl).

17. A method for simultaneous detection of dopamine and serotonin, the method comprising the following steps of:
(1) treating a biological sample isolated from an individual on the electrochemical sensor for simultaneous detection of dopamine and serotonin of claim 1; and
(2) performing differential pulse voltammetry (DPV) on the sample-treated sensor at pH 6 to 8.
